(19) United States
(12) Patent Application Publication
(10) Pub. No.: US 2007/0092573 A1
(43) Pub. Date: Apr. 26, 2007

(54) STABILIZED EXTENDED RELEASE PHARMACEUTICAL COMPOSITIONS COMPRISING A BETA-ADRENORECEPTOR ANTAGONIST

(76) Inventors: Laxminarayan Joshi, Toronto (CA); Robert Scott Lefler, Brantford (CA)

Correspondence Address: PEACOCK MYERS, P.C., 201 THIRD STREET, N.W., SUITE 1340, ALBUQUERQUE, NM, 87102, US

(21) Appl. No.: 11/551,865

(22) Filed: Oct. 23, 2006

Related U.S. Application Data
(63) Continuation-in-part of application No. 11/257,526, filed on Oct. 24, 2005.

(51) Int. Cl. A61K 31/205; A61K 9/14

(52) U.S. Cl. 424/487; 514/554

(57) ABSTRACT

The present invention is a new stable extended release drug composition particularly suitable for use as a beta-adrenoreceptor antagonist agent. The present invention is specifically a drug composition comprising a pharmaceutical, a methacrylic acid copolymer and a matrix forming agent, and a method for manufacturing same. When applied to highly soluble drugs like metoprolol succinate, the resulting drug composition is characterized by an extended-release profile.

### FIELD OF THE INVENTION

The present invention is a new stable extended release pharmaceutical composition for treating cardiovascular disorders, and more particularly a stable extended release pharmaceutical composition containing as an active substance, a beta-adrenoreceptor antagonist, and a method of preparing such composition.

### BACKGROUND OF THE INVENTION

Metoprolol succinate, a chemically synthesized compound, is known to act as a beta-adrenoreceptor antagonist. It is used to treat cardiovascular disorders, such as hypertension, in humans.

Metoprolol succinate is highly soluble, resulting in rapid dissolution and absorption. Accordingly, effective treatments using Metoprolol succinate ordinarily require large and frequent dosing. This, in turn, results in increased incidents of side effects, poorer patient compliance and higher costs. One way in which to minimize these problems is to provide for the extended release of a less soluble composition of the drug in the body.

The advantages of extended release products are well known in the pharmaceutical field and include improved clinical efficacy, reduced fluctuations in concentrations of the drug in the blood, cost effectiveness and increased patient compliance by reducing the number of administrations necessary to achieve the desired result. These advantages have been attained by a wide variety of methods, including methods to control dissolution, diffusion, swelling, osmotic pressure and ion exchange. These methods experience a variety of problems, and range in terms of cost and difficulty in delivery.

For example, different hydrogels have been described for use in controlled release medicines, some of which are synthetic, but most of which are semi-synthetic or of natural origin. A few contain both synthetic and non-synthetic material. However, some of the systems require special process and production equipment, and in addition some of these systems are susceptible to variable drug release.

Oral controlled release delivery systems should ideally be adaptable so that release rates and profiles can be matched to physiological and chronotherapeutic requirements. While many controlled and sustained-release formulations are already known, it is often not possible to readily predict whether a particular sustained-release formulation will provide the desired sustained release profile for a particular drug, and it has generally been found that it is necessary to carry out considerable experimentation to obtain extended release formulations of such drugs having the desired rate of release when ingested

An example of a controlled release delivery system is described by Dahlinder et al. (U.S. Pat. No. 4,927,649). This consists of a compact inert core of either glass or silicon dioxide covered by a layer of a pharmaceutically active compound, which in turn is covered by a polymeric membrane. The polymeric membrane dissolves to expose the drug in the gastric environment at rates determined by diffusion of fluid into the coated cores. This method of controlling and extending the release of a pharmaceutically active compound requires a sophisticated coating process and involves organic solvents that are corrosive and toxic and also requires sophisticated disposal techniques. Accordingly, this method is expensive, time consuming and non-environmentally friendly.

Another example of a controlled release delivery system is described by Ragnarsson et al (U.S. Pat. No. 4,942,040). This consists of coating beads of metoprolol with a water insoluble polymeric membrane, dispersing dihydropyridine in a non-ionic solubilizer, mixing the dihyrdopyridine with a dihydrophilic swelling agent to form a swollen gel matrix when it comes into contact with water and incorporating the coated metoprolol into the swollen gel matrix system. The swollen gel matrix systems prevent the rapid release of the drug while the coating on the beads of metoprolol protect the drug from rapid dissolution. However, the use of the swollen gel matrix results in a bulky product that is difficult to consume and contains small amounts of active ingredient. Accordingly, this method is not efficient and remains problematic.

Another example of a controlled release delivery system is described by Jonsson et al (U.S. Pat. No. 4,942,040). This consists of coating beads of metoprolol with a pH independent polymer, such as ethylcellulose. This method of controlling and extending the release of a pharmaceutically active compound requires a sophisticated coating process and involves organic solvents which are corrosive and toxic and also requires sophisticated disposal techniques. Accordingly, this method is expensive, time consuming and non-environmentally friendly.

Another example of a controlled release delivery system is described by Baichwal et al (U.S. Pat. No. 5,399,358). This consists of incorporating metoprolol into a gum based matrix formulation, preferably using xanthan gum and locust bean gum. As the gums slowly hydrate, the drug is released to provide an extended release formulation. However, this gum based matrix present microbiological problems and requires a complicated and expensive process to manufacture, requiring sophisticated machinery and skilled workers.

Accordingly, it is desirable to provide for an extended release pharmaceutical composition containing as an active substance, a beta-adrenoreceptor antagonist, and a method of preparing such composition, which solves the problems presented by the existing art.

### SUMMARY OF THE INVENTION

The present invention is a stabilized extended-release drug composition comprising a pharmaceutical, a methacryclic acid copolymer and a matrix forming agent.

The present invention further provides a method for manufacturing the above drug composition by granulating a pharmaceutical with a methacryclic acid copolymer and an alkalinizer solution to coating the granulated pharmaceutical with the methacryclic acid copolymer, adding a matrix forming agent and a basifier to the resulting mixture .

One embodiment of the present invention provides for a drug composition comprising a pharmaceutical, a methacryclic acid copolymer and a matrix forming agent. For example, the pharmaceutical can be a beta-adrenoreceptor antagonist. The methacryclic acid copolymer can be a Eudragit® methacryclic acid copolymer. The matrix forming agent can be a Carbopol® polyacrylic acid copolymer.

Another embodiment of the present invention provides for a drug composition comprising the beta-adrenoreceptor antagonist metoprolol succinate. The matrix forming agent of a Carbopol® polyacrylic acid copolymer can be enhanced by the use of a poly-oxide compound, such as a Polyox® polyethylene oxide compound. The release profile of the matrix can be controlled by the use of a basifier, such as di-calcium phosphate.

Yet another embodiment of the present invention provides for a method for manufacture of a drug composition. The method includes mixing together a pharmaceutical active ingredient, such as metoprolol succinate, a methacryclic acid copolymer such as a Eudragit® methacryclic acid copolymer and microcrystalline cellulose. This mixture is granulated with a solution of an alkalinizer such as sodium bi-carbonate and water. The granulated mass is dried and sized. Matrix forming agents such as a Carbopol ® polyacrylic acid copolymer, a Polyox® polyethylene oxide compound and a Eudragit methacrylic acid copolymer, are added to the mixture in addition to a basifier such as di-calcium phosphate and a lubricant such as magnesium stearate. The mixture can be formed into tablets that are covered with a hypromellose based coating, titanium dioxide and a plasticizer such as polyethylene glycol.

### BRIEF DESCRIPTION OF THE DRAWINGS

FIGS. 1A and 1B are illustrations of a stabilized extended release pharmaceutical composition in a non-eroding matrix formulation in relaxed and swollen forms, respectively.

FIG. 2 is a table showing the dissolution of sample capsules as compared to a control.

### DETAILED DESCRIPTION

Metoprolol Succinate is a highly water-soluble compound and the absorption of metoprolol is rapid and complete in humans. Plasma levels following oral administration of conventional metoprolol tablets approximate 50% of levels following intravenous administration, indicating about 50% first-pass metabolism. Elimination is mainly by biotransformation in the liver, and the plasma half-life ranges from approximately 3 to 7 hours. Less than 5% of an oral dose of metoprolol is recovered unchanged in the urine and the remaining 45% is excreted by the kidneys as clinically insignificant metabolites. Only a small fraction of the drug, about 12%, is bound to human serum albumin. The combination of the factors of high solubility and short half-life has required large and frequent dosing for effective treatment with metoprolol succinate. However, such treatment results in toxicity and compliance problems, as well as increased incidence of side effects.

Decreasing the solubility of metoprolol succinate will help resolve the problem of toxicity associated with large and frequent dosing. It is possible to decrease the solubility of metoprolol succinate by coating a granulation of the drug with a methacrylic acid co-polymer, such as a Eudragit® methacryclic acid copolymer, that does not dissolve in a solution with low pH, such as solutions with pH lower than about 6.0 to 7.0, but will dissolve in a solution with high pH, such as solutions with pH greater than about 6.0 to 7.0. While a Eudragit® methacryclic acid copolymer has been used as enteric and moisture coating, it is found that it can be melted and used to coat granulations of drugs and when applied in this manner it has the effect of decreasing solubility and protecting the drug it is applied to from rapid dissolution and absorption. However, since it is preferable to resolve all of the problems associated with large and frequent dosing, it is not sufficient to decrease the solubility of metoprolol succinate without also providing for an extended release of the drug.

In comparison to conventional metoprolol succinate treatments, the plasma metoprolol levels following administration of extended release metoprolol succinate are characterized by lower peaks, longer time to peak and significantly lower peak to trough variation. The peak plasma levels following once daily administration of extended release metoprolol succinate average one-fourth to one-half the peak plasma levels obtained following a corresponding dose of conventional metoprolol, administered once daily or in divided doses. At steady state the average bioavailability of metoprolol following administration of extended release metoprolol succinate, across the dosage range of 50 to 400 mg once daily, was 77% relative to the corresponding single or divided doses of conventional metoprolol. Nevertheless, over the 24 hour dosing interval, b₁-blockade is dose-related and comparable to the non-extended dosage form. Extended release metoprolol succinate shows an increase in bioavailability that is proportional, although not directly, to increase in dosage, which is not significantly affected by stomach contents.

It is desirable that the method used to provide for the extended release profile of metoprolol succinate results in a composition yielding a release profile over a period of approximately 24 hours, while avoiding the problems associated with coating beads of the drug, swollen gel systems, organic solvents and gum based systems. The present invention is able to resolve the problems associated with these methods by first utilizing a novel method of granulation in which the drug particles are granulated with a coating material and then prepared in a non-eroding matrix formulation with matrix controlling polymers. By utilizing this method, an extended release composition can be prepared which provides for a release profile of approximately 24 hours that requires less sophisticated equipment, technology and skill, is less expensive, safer and non-toxic to prepare, provides a treatment that is easy to use while containing the appropriate amount of the drug, is environmentally friendly, is free from microbiological problems and is not substantially affected by the quantity or composition of the gastric fluid.

An additional characteristic of the present invention is that the release profile can be adjusted by controlling the rate of fluid penetrating into the tablet core. The viscosity of the matrix is an essential factor affecting the rate of fluid penetrating into the tablet core. The viscosity of the matrix is inversely proportional to the rate of the release of the drug from the matrix. The viscosity of the matrix is determined by the viscosity of the matrix forming agents, such as a Carbopol® polyacrylic acid copolymer, a Polyox® polyethylene oxide compound and a Eudragit® methacryclic acid copolymer that does not dissolve in a solution having a pH not less than about 5.0, but that does swell in a solution have a pH of about 5.0 and greater. A Polyox® polyethylene oxide compound is chemically known as polyethylene oxide and is a water soluble resin or polymer, has a molecular weight of about 6 million and yields a high viscosity solution in water. A Carbopol® polyacrylic acid copolymer is a polyacrylic acid copolymer that is insoluble in water and achieves its maximum viscosity in environments where the pH level is basic. Some methacryclic acid copolymers, such as some Eudragit® methacryclic acid copolymers, for example Eudragit® EPO, do not dissolve in a solution having a pH not less than about 5.0, but do swell in a solution have a pH of about 5.0 and greater. The viscosity of such Eudragit® methacryclic acid copolymers and Carbopol® polyacrylic acid copolymers is directly proportional to the pH of their environment. Accordingly, a basifier, such as di-calcium phosphate, is utilized in proportion to the amount of the Eudragit® methacryclic acid copolymer and the Carbopol® polyacrylic acid copolymer in the matrix, depending on the desired release profile.

In a preferred embodiment of the present invention, a pharmaceutical beta-adrenoreceptor antagonist (for example, metoprolol succinate) is granulated and coated with a methacrylic acid copolymer, such as a Eudragit® methacryclic acid copolymer. Methacrylic acid copolymers have been used as an enteric coating for dosage formulations to mask the undesirable taste associated with some formulations and also as a protective coating against the acidic environment of the stomach for those molecules that degrade in acidic environment of the stomach (i.e delayed release coating or enteric coating). However, it has been discovered that methacrylic acid copolymers decrease the solubility of the drug that it coats when applied to granulated pharmaceuticals such as metoprolol succinate, thus slowing the dissolution of the pharmaceutical. An alkalinizer, such as sodium bi-carbonate, is used to melt the methacrylic acid copolymer in order to apply it to the granulated pharmaceutical. The coated granules of the pharmaceutical are then prepared in a non-eroding matrix formulation, comprised of a poly acrylic compound such as a Carbopol® polyacrylic acid copolymer, a poly-oxide compound such as a Polyox® polyethylene oxide compound and a methacrylic acid copolymer, such as a Eudragit® methacrylic acid copolymer, to prevent the coated granules from passing through the stomach too quickly. A basifier, such as di-calcium phosphate, can be used in the matrix formulation to control the release profile. The resulting mixture can be formed into tablets and coated with a hypromellose based coating, titanium dioxide and a plasticizer, such as Spectrablend White®. This results in a pharmaceutical composition providing the extended release of the pharmaceutical over the period of approximately 24 hours when the dosage form is exposed to an environmental fluid.

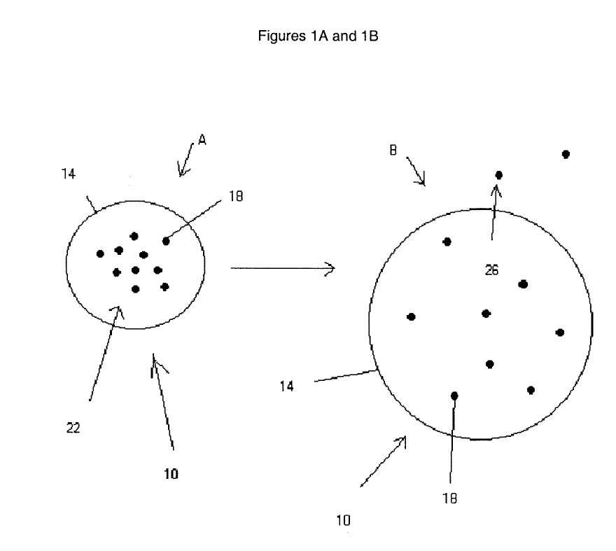

FIGS. 1 A and 1B show a stabilized extended release pharmaceutical composition (10) in a non-eroding matrix formulation (14) in relaxed and swollen forms, respectively. When a dosage form containing a drug (18) (e.g. beta-adrenoreceptor antagonist agent) in a matrix formulation (14) is ingested and exposed to a gastric environment (FIG. 1A), dissolution material, such as gastric fluids (22), enters into the tablet matrix (14) causing the form to swell to capacity (FIG. 3B), preventing rapid release of the drug (18). During the initial period following exposure, leeching (26) of drug (18) from the swollen tablet matrix (FIG. 1B) occurs. This allows for the commencement of the therapeutic effects of the drug (18) without delay. This release mechanism continues over an extended period providing the desired extended release profile.

Manufacture of a preferred embodiment of the present invention is achieved using the following steps (which are provided for example purposes only):

| Number | Step |
|---|---|
| 1. | mix together metoprolol succinate, Eudragit S 100 ® |
| | and Microcrystalline cellulose |
| 2. | dissolve sodium bi-carbonate in water to form a solution; |
| 3. | use the solution from step 2 to granulate the |
| | resulting mixture of step 1; |
| 4. | dry the granulated mass and size the granules; |
| 5. | add Polyox WSR 303 ®, Carbopol 71G ® and |
| | Dicalcium Phosphate to the granules obtained in step 4; |
| 6. | add Magnesium stearate as a lubricant; |
| 7. | form the resulting mixture into tablets; |
| 8. | coat the tablets with hypromellose, titanium |
| | dioxide and polyethylene glycol. |

In furtherance of the example above, the following dosages of metoprolol succinate can be manufactured using the following amounts of the listed ingredients:

### EXAMPLE 1

| | Quantitative | |
|---|---|---|
| Name of the ingredients | composition | |
| Metoprolol Succinate | 23.75 | mg |
| Methacrylic acid copolymer | 16.25 | mg |
| (Eudragit S 100 ®) | | |
| Microcrystalline cellulose 12 | 27.375 | mg |
| Sodium Hydrogen Carbonate | 02.500 | mg |
| Polyethylene oxide | 12.500 | mg |
| (Polyox WSR 303 ®) | | |
| Carbomera (Carbopol 71 G ®) | 11.875 | mg |
| Methacrylic acid copolymer | 02.500 | mg |
| (Eudragit EPO ®) | | |
| Calcium Hydrogen Phosphate | 07.625 | mg |
| dihydrate(unmilled) | | |
| Magnesium Stearate | 08.625 | mg |
| Purified Water for granulation | 0.0527 | ml |
| Opadry White | 03.000 | mg |
| Water | 0.0250 | ml |

### EXAMPLE 2

| | Quantitative | |
|---|---|---|
| Name of the ingredients | composition | |
| Metoprolol Succinate | 95.00 | mg |
| Methacrylic acid copolymer | 65.000 | mg |
| (Eudragit S 100 ®) | | |
| Microcrystalline cellulose 12 | 109.50 | mg |
| Sodium Hydrogen Carbonate | 10.000 | mg |
| Polyethylene oxide | 50.000 | mg |
| (Polyox WSR 303 ®) | | |
| Carbomera (Carbopol 71 G ®) | 57.500 | mg |
| Methacrylic acid copolymer | 10.000 | mg |
| (Eudragit EPO ®) | | |
| Calcium Hydrogen Phosphate | 33.600 | mg |
| dihydrate(unmilled) | | |
| Magnesium Stearate | 21.400 | mg |
| Purified Water for granulation | 0.2108 | ml |
| Opadry White | 12.000 | mg |
| Water | 0.100 | ml |

### EXAMPLE 3

| | Quantitative | |
|---|---|---|
| Name of the ingredients | composition | |
| Metoprolol Succinate | 95.00 | mg |
| Methacrylic acid copolymer | 65.000 | mg |
| (Eudragit S 100 ®) | | |
| Microcrystalline cellulose 12 | 109.50 | mg |
| Sodium Hydrogen Carbonate | 10.000 | mg |
| Polyethylene oxide | 50.000 | mg |
| (Polyox WSR 303 ®) | | |
| Carbomera (Carbopol 71 G ®) | 57.500 | mg |
| Methacrylic acid copolymer | 10.000 | mg |
| (Eudragit EPO ®) | | |
| Calcium Hydrogen Phosphate | 33.600 | mg |
| dihydrate(unmilled) | | |
| Magnesium Stearate | 21.400 | mg |
| Purified Water for granulation | 0.2108 | ml |
| Opadry White | 12.000 | mg |
| Water | 0.100 | ml |

### EXAMPLE 4

| | Quantitative | |
|---|---|---|
| Name of the ingredients | composition | |
| Metoprolol Succinate | 190.00 | mg |
| Methacrylic acid copolymer | 130.000 | mg |
| (Eudragit S 100 ®) | | |
| Microcrystalline cellulose 12 | 219.00 | mg |
| Sodium Hydrogen Carbonate | 20.000 | mg |
| Polyethylene oxide | 100.000 | mg |
| (Polyox WSR 303 ®) | | |
| Carbomera (Carbopol 71 G ®) | 115.000 | mg |
| Methacrylic acid copolymer | 20.000 | mg |
| (Eudragit EPO ®) | | |
| Calcium Hydrogen Phosphate | 67.200 | mg |
| dihydrate(unmilled) | | |
| Magnesium Stearate | 42.800 | mg |
| Purified Water for granulation | 0.4036 | ml |
| Opadry White | 24.000 | mg |
| Water | 0.200 | ml |

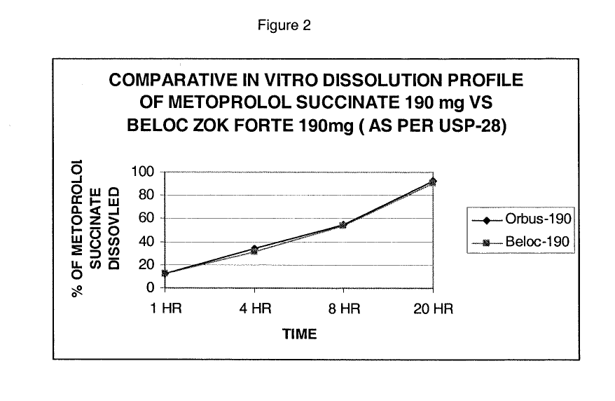

Sample capsules containing metoprolol succinate as the active ingredient were prepared according to the above Example 4 and were subject to in vitro dissolution studies. It was found that the comparative in vitro dissolution of the sample capsules with respect to Beloc®, used as a control, was equivalent, as shown in FIG. 2.

While the subject invention has been described and illustrated with reference to certain particular embodiments thereof, those skilled in the art will appreciate that various adaptations, changes, modifications, substitutions, deletions or additions of procedures and protocols may be made without departing from the scope of the invention.

What is claimed is:

1. A drug composition comprising:
a pharmaceutical;
a coating; and
a matrix forming agent.

2. The drug composition of claim 1 wherein the pharmaceutical is a beta-adrenoreceptor antagonist.

3. The drug composition as claimed in claim 2 wherein the beta-adrenoreceptor antagonist is metoprolol succinate.

4. The drug composition as claimed in claim 1 wherein the coating is a methacryclic acid copolymer;

5. The drug composition as claimed in claim 4 wherein the methacryclic acid copolymer dissolves in a solution with a pH not less than about 6.0 to 7.0.

6. The drug composition as claimed in claim 1 wherein the matrix forming agent is a poly acrylic compound.

7. The drug composition as claimed in claim 6 wherein the poly acrylic compound is a poly acrylic acid copolymer.

8. The drug composition as claimed in claim 7 wherein the matrix forming agent further includes a polyethylene-oxide compound.

9. The drug composition as claimed in claim 8 wherein the poly-oxide compound is polyethylene oxide having a molecular weight greater than 1,000,000 amu.

10. The drug composition as claimed in claim 9 wherein the matrix forming agent further includes a methacrylic acid copolymer that does not dissolve in a solution with a pH not less than about 5.0.

11. The drug composition as claimed in claim 1 further comprising a lubricant and a filler.

12. The drug composition as claimed in claim 5 further comprising an alkalinizer.

13. The drug composition as claimed in claim 12 wherein the alkalinzer is sodium bi-carbonate.

14. The drug composition as claimed in claim 7 further comprising a basifier.

15. The drug composition as claimed in claim 14 wherein the basifier is di-calcium phosphate.

16. The drug composition as claimed in claim 15 wherein the pharmaceutical is a beta-adrenoreceptor antagonist.

17. The drug composition as claimed in claim 16 wherein the beta-adrenoreceptor antagonist is metoprolol succinate.

18. The drug composition as claimed in claim 17 wherein the coating is a methacryclic acid copolymer;

19. The drug composition as claimed in claim 18 wherein the methacryclic acid copolymer dissolves in a solution with a pH not less than about 6.0 to 7.0.

20. The drug composition as claimed in 19 further comprising an alkalinizer.

21. The drug composition as claimed in 20 wherein the alkalinizer is sodium bicarbonate.

22. The drug composition as claimed in claim 21 wherein the matrix forming agent further includes a poly-oxide compound.

23. The drug composition as claimed in claim 22 wherein the poly-oxide compound is polyethylene oxide having a molecular weight greater than 1,000,000 amu.

24. The drug composition as claimed in claim 23 wherein the matrix forming agent further includes a methacrylic acid copolymer that does not dissolve in a solution with a pH not less than about 5.0.

25. The drug composition as claimed in claim 24 further comprising a lubricant and a filler

26. A drug composition comprising:
a beta-adrenoreceptor antagonist;
a methacryclic acid copolymer;
an alkalinizer;
a matrix forming agent comprising a poly acrylic compound;
a matrix forming agent further comprising a poly-oxide compound; and
a basifier

27. A method for manufacture of a drug composition comprising:
mixing a pharmaceutical, a methacrylic acid copolymer and a filler;
dissolving an alkalinizer in water to form a solution;
granulating the mixture with the solution to form a resulting mixture;
drying the resulting mixture and sizing the granules.
adding a matrix forming agent to the dried mixture;
adding a basifier to the dried mixture; and
adding a lubricant to the dried mixture.

28. The method as claimed in claim 27 wherein the pharmaceutical is a beta-adrenoreceptor antagonist.

29. The method as claimed in claim 28 wherein the beta-adrenoreceptor antagonist is metoprolol succinate.

30. The method as claimed in claim 27 wherein the methacryclic acid copolymer is dissolves in a solution with a pH not less than about 6.0 to 7.0.

31. The method as claimed in claim 27 wherein the filler is chosen from the group consisting of microcrystalline cellulose and sorbitol.

32. The method as claimed in claim 27 wherein the lubricant is magnesium stearate.

33. The method as claimed in claim 29 wherein the matrix forming agent is a poly acrylic compound.

34. The method as claimed in claim 33 wherein the poly acrylic compound is a poly acrylic acid copolymer.

35. The method as claimed in claim 34 wherein the matrix forming further comprises a polyethylene-oxide compound.

36. The method as claimed in claim 33 wherein and the polyethylene-oxide compound is polyethylene oxide having a molecular weight greater than 1,000,000 amu.

37. The drug composition as claimed in claim 36 wherein the matrix forming agent further includes a methacrylic acid copolymer that does not dissolve in a solution with a pH not less than about 5.0.

38. The method as claimed in claim 37 wherein the alklinizer is sodium bicarbonate.

39. The method as claimed in claim 38 wherein the basifier is di-calcuim phosphate.

40. The method as claimed in claim 27 further comprising:
forming the resulting mixture into tablets

41. The method as claimed in claim 40 further comprising:
applying a hypromellose based coating, titanium dioxide and a plasticizer to the tablets.
